(19) United States
(12) Patent Application Publication
(10) Pub. No.: US 2015/0219542 A1
(43) Pub. Date: Aug. 6, 2015

(54) DEVICE FOR HOME MONITORING OF HAEMATOLOGICAL PARAMETERS OF PATIENTS

(71) Applicant: KONINKLIJKE PHILIPS N.V., EINDHOVEN (NL)

(72) Inventor: Nicholas Young Kent, Sandhurst (GB)

(21) Appl. No.: 14/419,618

(22) PCT Filed: Aug. 10, 2013
(86) PCT No.: PCT/IB2013/056551

§ 371 Date: Feb. 4, 2015

Related U.S. Application Data
(60) Provisional application No. 61/681,181, filed on Aug. 9, 2012.

(51) Int. Cl. G01N 15/10; A61B 5/00; G06F 19/00; A61B 5/01

(52) U.S. Cl. CPC G01N 15/10; A61B 5/01; A61B 5/742; A61B 5/747; A61B 5/0015; G06F 19/3406; G06F 19/363; G01N 2015/1062; G01N 2015/008

(57) ABSTRACT

The present invention relates to a device for home monitoring of haematological parameters of patients. In particular, the device may be configured for home monitoring of patients undergoing chemotherapy. A white blood cell counter is presented in combination with a physically separated user device. The user interface and the white blood cell counter are configured to exchange data with each other. The present invention allows the white blood cell counter to be produced at lower costs, since no display is integrated into it directly. The patients may use the user interface device anywhere in their home or even on the go, allowing the maximum in freedom to fill in patient-reported outcomes or health assessment questionnaires. Also taking temperature readings is facilitated. In the home, a white blood cell test can be performed if required. The user interface guides the patient through the correct process and may therefore be brought to the cell counter.

### FIELD OF THE INVENTION

The present invention relates to the field home monitoring, in particular home monitoring of haematological parameters such as those used in the field of chemotherapy patients. In particular, the present invention relates to a device for home monitoring of haematological parameters of patients and a method of home monitoring of haematological parameters of patients.

### BACKGROUND OF THE INVENTION

According to the state of the art equipment, white blood cell counting readers are stationary, hospital-based devices which are for professional use only. Further, most chemotherapy regimens are myelosuppressive, meaning that they inhibit the bone marrow activity, resulting in decreased production of blood cells and platelets. After receiving chemotherapy, patients with low blood cells counts are in danger of serious complications in form of an infection as well as not being able to receive a next treatment due to low counts.

Furthermore, there are other disease and treatments that benefit from regular blood cell counting, like for example chronic obstructive pulmonary disease (COPD), warfarin treatment for anti-coagulation therapy and autoimmune disease, such as rheumatoid arthritis.

### SUMMARY OF THE INVENTION

There may be a need to provide for home monitoring of patients undergoing chemotherapy.

The object of the present invention may be seen as to provide for an effective and safe manner of home monitoring of patients which are currently undergoing chemotherapy.

The object of the present invention is solved by the subject-matter of the independent claims. Further embodiments and advantages of the invention are incorporated in the dependent claims.

The described embodiments similarly pertain to the device f for home monitoring of haematological parameters of patients and the method of home monitoring of haematological parameters of patients. Synergetic effects may arise from different combinations of different embodiments, although they might not be described in detail hereinafter.

Furthermore, it shall be noted that all embodiments of the present invention concerning a method might be carried out with the order of the steps as described, nevertheless this has not to be the only and essential order of the steps of the method of the present invention. The skilled person gathers all kinds of different orders and combinations of the method steps described herein, unless explicitly mentioned to the contrary hereinafter.

According to an exemplary embodiment of the invention a device for home monitoring of haematological parameters is presented. The device comprises a white blood cell counter and a user interface. Furthermore, the white blood cell counter is configured for receiving a sample probe of the patient and to determine a value of white blood cells in the received sample probe. The user interface is physically and/or structurally separated or separable from the white blood cell counter. Furthermore, the user interface is configured to display information based on the value of white blood cells in the received sample probe as determined by the white blood cell counter. The white blood cell counter may be portable. However, its portability is not essential.

In the context of the present invention the term “physically and/or structurally separated or separable” shall be used in the following sense. The present invention provides for two physically different mechanical entities, namely the WBC counter and the user interface. These entities are accommodated in individual housings. Thus, the user interface is not integrally formed with the WBC counter. Nevertheless, according to some embodiments of the invention, it can be connected and reconnected to the WBC counter. The idea is that the user interface can used in a location different than that of the white blood cell counter. Moreover, as will become apparent from and elucidated with reference to the embodiments described hereinafter, the user interface and the WBC counter of the present invention may be connected via a mechanical and/or an electronical and/or wireless connection. This will be described hereinafter in more detail.

Herein the term “sample probe” may be understood as sample test cartridge by means of which body fluids of the patient, like blood, can be stored and can be introduced into the WBC counter. Such sample probes or sample test cartridges may also be part of the presented device and can used in the context of the presented method. The sample probe may be seen as means for providing the WBC counter with a blood probe of the patient.

Therein, the physical separation of the user interface from the white blood cell (WBC) counter may also be seen as a structural separation. Consequently, two different devices are presented with the WBC counter and the user interface. If desired, the user interface may have another additional or a plurality of additional functionalities which increase the flexibility of the user interface. Such additional functionalities will be described in detail hereinafter.

In general the present invention is directed to the home monitoring of haematological parameters. As will be described in the following different applications may benefit from the presented devices and methods of the present invention. The presented device may be especially applicable for home monitoring of patients undergoing chemotherapy but may also be adapted for home monitoring of patients of chronic obstructive pulmonary disease (COPD), warfarin treatment for anti-coagulation therapy and autoimmune disease, such as rheumatoid arthritis. The embodiment of the present invention presented above may beneficially used for these applications as the advantages of the structurally separated user interface and the WBC counter mutually apply here. However, in the following the present invention will be described in relation to patients having cancer, which shall not be construed as limiting the present invention.

The presented device can be used for monitoring of patients undergoing chemotherapy where the three parameters of white blood cell counts, body temperature of the patient and patient reported outcomes or health assessment questionnaires are used as monitoring parameters. Therein the presented device provides for the following advantages. Due to the separation of the user interface from the WBC counter, the user interaction is allowed away from the WBC counter, for example when taken a temperature or completing a questionnaire. The communication channel between the WBC counter and the user interface may be unidirectional or bidirectional. The WBC counter may be seen as a home test device.

The inventors of the present invention found out that patients prefer either the kitchen or the bathroom for a WBC counter. The patient-reported outcomes (PRO) self-assessment can take place anywhere in the home, or even on the move by means of the presented user interface. If the user interface would be bound tightly to the white blood cell (WBC) counter, then the patient would not be able to use the device as planned. However, the present invention provides for an advantageous solution by separating the user interface from the WBC counter. In addition, the user interface can be connected to the WBC counter mechanically or electronically via a wire-less connection. Furthermore, the clinician might set a schedule for each parameter to be monitored that requires less frequent WBC measurements than other parameters, e. g. through a schedule identified by the WBC trajectory modelling or on-demand, when the patient feels unwell, meaning that a more suitable location for the interaction is needed, e.g. bedside or mobile. The presented device matches these needs and allows the necessary communication via the user interface from the corresponding suitable location.

In the following several different details about the user interface are described which may be embodied in the presented device alone or in combination with other features described herein. The user interface may be seen as the intelligence of the presented device. Thus, the user interface may be seen as a telehub of the presented system due to various communication and/or calculating functionalities. The user interface may transmit temperature values of the patient received from a thermometer and/or results of the WBC counts received from the WBC counter and/or a completed questionnaire via e.g. GPRS to a server, or, if desired. These data may also be forwarded directly into a hospital record system. Further, the clinician may instruct to perform a non-scheduled test.

The user interface may have stored on it several different intervention levels regarding a predetermined temperature value or regarding a predetermined WBC result value. The user interface may also be configured to transmit received temperature values and/or WBC values as soon as it receives such values, i.e. results. The user interface may also be configured to transmit these results to a remote server which forwards these results or values to the clinician. The user interface may also be configured to transmit the completed questionnaire to the clinician via server. Also a combined data set of temperature, WBC result and completed questionnaire may be transmitted. Further, the user interface may be configured to compare received values with the stored respective intervention levels and may generate a signal for the patient in case an undesired situation is detected based on the comparison. For example, a message may be displayed on the user interface which reads “Contact your clinician”. Also sound signals may be used. Further, an email and/or a sms may be sent by the user interface via e.g. GPRS to a clinician, informing him about the current situation of the monitored patient. If desired another embodiment is possible described in the following. The user interface, i.e. the telehub, may send information, like the temperature values of the patient received from a thermometer and/or results of the WBC counts received from the WBC counter and/or a completed questionnaire via GPRS to the server. Further, the server sends an email and/or a sms to the clinician. This is part of the system according to an exemplary embodiment described herein and which comprises the device for home monitoring of haematological parameters and the server.

In any embodiment described herein, the user interface may be configured to send only if the comparison has detected an undesired situation of the patient. Also other data and/or communication networks may be used by the user interface like e.g. Bluetooth, Wireless Local Are Network, UMTS, 3G, or others. For guiding the user through a WBC count process it may comprise a program element by means of which the functionalities of the WBC reader may be controlled remotely or in mechanical contact with the WBC reader, i.e. the WBC counter. This will be explained with more detail hereinafter.

Thus, the presented device for home monitoring of haematological parameters or for home monitoring of patients undergoing chemotherapy can be safely and reliably be used by patients on their own. The presented device, in particular the user interface, is configured to carry out a method of communicating with the patient, informing the patient about upcoming tests that he needs to perform, like WBC counting or temperature measurement. This allows the patient to complete the questionnaire and feedback any other information from the clinician to the patient. More details and advantages will be described with respect to several embodiments of the present invention hereinafter.

The presented separation of the user interface from the WBC counter provides for another advantage. Often, patients undergoing treatment for cancer, require additional treatments on top of their chemotherapy, for example radiotherapy or hormone therapy. In some cases it can be sufficient after chemotherapy to only monitor the temperature and patient reported outcomes, which can be done by the user interface alone. Thus, the presented solution allows the WBC reader to be removed and to be reconditioned after the first treatment (of chemotherapy) and the separate user interface to be left with the patient during subsequent therapies. This may significantly reduce costs during a home monitoring process.

According to another exemplary embodiment the user interface is configured to transmit a questionnaire, which has been completed by the user, to a remote server.

Therein, the server may forward the questionnaire to the clinician who is responsible for this individual patient.

According to another exemplary embodiment, the device comprises a thermometer which is configured to determine a value of a body temperature of the patient. Furthermore, the thermometer and the user interface are configured to provide the determined value of the body temperature to the user interface.

In other words, user interface and the thermometer are configured to exchange data and/or communicate with each other. The communication channel may be unidirectional or bidirectional. There may be various embodiments of the configuration of the thermometer and/or the user interface, such that the determined temperature is provided to the user interface. For example, the user interface may be configured to frequently and electronically read out the thermometer via for example short-range local radiofrequency communication or also via for example a wide-range radiofrequency communication. Also infrared or Bluetooth may be used for transmitting the determined temperature from the thermometer to the user interface. In an analogue way, the thermometer may be configured to frequently transmit, i.e. to send, the determined temperature to the user interface. Certainly, the above applies in the same way for a plurality of determined temperature values. The user interface may be provided with a temperature grasp functionality by means of which the user may trigger the grasping process causing the transmission of the value of the temperature to the user interface. However, also a purely automatic procedure may be implemented in the user interface.

According to another exemplary embodiment of the invention, the thermometer comprises an interface, in particular a Bluetooth interface, for wireless communication with the user interface.

Thus, the thermometer may comprise a Bluetooth sender and interface and the user interface may comprise a Bluetooth receiver. Further, the thermometer may be configured to carry out three different temperature measurements per inquiry to get rid of measurement fluctuations. If desired, the thermometer may be configured to transmit only the highest of the three measured temperatures. Of course also other numbers, like two, four five, six or more, of different temperature measurements per inquiry are possible. Therein, it is also possible that the thermometer and the WBC counter are configured for a respective change of data between each other. It may thus be an embodiment of the present invention that all elements the WBC counter, the user interface and the thermometer are configured to exchange data with each other via unidirectional or also bidirectional data transmission. In a further embodiment all three measurements are sent to the user interface and the user interface will then use the highest of these values for onward transmission to the server.

According to another exemplary embodiment of the invention, the white blood cell counter and the user interface are configured to provide the determined value of white blood cells in the received sample probe to the user interface.

According to another exemplary embodiment of the invention the user interface stores a white blood cell intervention level and a temperature intervention level. Furthermore, the user interface is configured for comparing the determined value of the body temperature, which was received from the thermometer with the temperature intervention level stored in the user interface. Moreover the user interface is configured for comparing the determined value of white blood cells in the received sample probe with the white blood cell intervention level.

Said temperature intervention level and/or white blood cell intervention may be seen as stored and predetermined value, like a reference value, which is comprised by the user interface. In particular, the clinician may initially set up the user interface with respect to the individual patient which is monitored and with respect to the appropriate intervention levels. Furthermore, the user interface may comprise a health assessment questionnaire which is initially adapted to the individual patient by the clinician. For such storage the user interface may comprise an electronical storage medium, like e.g. a hard disk drive or solid state disk, or the like.

According to another exemplary embodiment of the invention the user interface is configured for generating a signal based on a result of the comparison, and wherein the signal is chosen from the group comprising a visual alert for the patient, an optical alert for the patient, an sms to a clinician, an email to a clinician, an alert message to a server, and any combination thereof.

Thus, the user interface may comprise a display and/or a loud speaker. Vibration means may also be applied.

According to another exemplary embodiment of the invention, the user interface is configured to receive data via an input of the patient. Furthermore, the device is configured to combine the body temperature received from the thermometer and the data received via inputs of the patient.

The configuration of the device to combine these data may be beneficially used in the context of health assessment questionnaires. In particular, the combined values may be entered into such a questionnaire or may also be submitted to a clinician or a server which will be described hereinafter in more detail. Therein, input as described above may be answers in the questionnaire during the home monitoring process.

According to another exemplary embodiment of the invention, the user interface comprises a display and an electronical storage medium. Furthermore, health assessment questionnaire for the patient is stored in the electronical storage medium for displaying the health assessment questionnaire to the patient by the display.

The storage medium may be seen as for example a USB stick, a data storage device, a hard disc, or any other medium on which information like a questionnaire can be stored. This embodiment ensures that the three parameters of white blood cell counts, temperature, and patient-reported outcomes or health assessment questionnaires are the basis for the home monitoring of the chemotherapy patients. Via external data transmission or communication channels to a clinician or a server communication with the patient is facilitated. Thus, the patient may be informed about upcoming tests he needs to perform. Hence, the presented device is configured to display to the user whether a white blood cell count or a temperature measurement has to be carried out.

Therefore, according to another exemplary embodiment of the invention the user interface may provide for a calendar and/or reminder function to remind the user which test is or which tests are due.

In parallel to the tests like temperature measurement and WBC counting, the user may complete a stored questionnaire via for example a touch screen. Subsequently, the completed questionnaire may be submitted via external data transmissions or communication channels by the device, e.g. by the user interface and/or by the WBC counter, to a server and then to a clinician.

According to another exemplary embodiment of the invention, the user interface is configured to transmit information to a remote server.

In other words, the presented device may a part of an interactive home monitoring system taking into account white blood cell counting, temperature measurement and patient-reported outcomes in health assessment questionnaire.

According to another exemplary embodiment of the invention, the WBC counter does not comprise a display. In other words, the white blood cell counter is a display-less device. This allows the white blood cell counter to be produced at lower costs.

According to another exemplary embodiment of the invention, the white blood cell counter comprises a first mechanical docking connector for receiving the user interface, and the user interface comprises a second mechanical docking connector for establishing a connection between the user interface and the white blood cell counter.

Therein, the first and the second mechanical docking connectors correspond to each other such that an engagement of the first and the second docking connectors is facilitated. Both docking connectors are configured such that an attachment or fixation of the user interface at the WBC counter is facilitated. In a further development, the mechanical docking connectors may comprise electrical connectors, such that a charging of the user interface may be facilitated by connecting it to the cell counter. Also data transmission may be facilitated via data transmission lines which are connected via the docking connectors.

According to another exemplary embodiment of the invention white blood cell counter comprises a first mechanical docking connector for establishing a connection with the user interface, and wherein the user interface comprises a second mechanical docking connector for establishing a connection with the white blood cell counter.

In general, the two mechanical docking connectors are mechanical interface for establishing a mechanical connection. The two mechanical docking connectors may be embodied for example as interfaces for connecting the WBC counter with the user interface via a wired connection. For example a kind of socket at the WBC counter and a kind of plug at the user interface may be practical embodiments thereof. However, also other physical connections between the WBC counter and the user interface are possible. If desired, the connection between the WBC counter and the user interface is established by a direct engagement between the two mechanical docketing connectors. But also another component, like e.g. a wire, may be used in between. As described herein in detail, also wire-less connections between the user interface and the WBC counter can be used alternatively or additionally to the wire-bound or other mechanical connections.

According to another exemplary embodiment of the invention, the user interface and the WBC counter are configured to exchange data with each other such that the user interface facilitates guiding the patient to perform a white blood cell counting on the WBC counter. In other words, in this configuration, the user interface may be seen as a control of the WBC counter. The user interface facilitates a control of the WPC reader by means of the user interface. Thus, patient may input several commands such that in the required steps, respective control commands are submitted from the user interface to the WBC counter.

According to another exemplary embodiment of the invention a method of home monitoring of patients is presented. The method comprises the step of determining a value of white blood cells of a sample probe of the patient by a white blood cell counter. Transmitting the determined value of white blood cells from the white blood cell counter to a physically separated user interface is another step of the method. Furthermore, monitoring the patient by the user interface based on the transmitted value of white blood cells by the user interface is a further step.

According to another exemplary embodiment of the invention the method comprises the step of receiving a value of a body temperature of the patient by the user interface from a thermometer.

According to another exemplary embodiment of the invention the user interface stores a white blood cell intervention level and a temperature intervention level and the user interface compares the provided, determined value of the body temperature with the temperature intervention level. The user interface further compares the determined value of white blood cells in the received sample probe with the white blood cell intervention level by the user interface.

According to another exemplary embodiment of the invention the step of generating a signal based on a result of the comparison by the user interface is comprised. Further, the signal is chosen from the group comprising a visual alert for the patient, an optical alert for the patient, a sms to a clinician, an email to a clinician, an alert message to a server, and any combination thereof.

It may be seen as a gist of the invention to provide for a WBC counter in combination with a structurally separated user interface that may communicate with the counter. By providing a thermometer, which is integrated into the communication between the user interface and the blood cell counter, a high degree of flexibility is provided for the user during home monitoring of patients. The present invention facilitates a communication between the WBC counter, the user interface, and the thermometer in any direction. The presented device may be connectable to a server.

According to another exemplary embodiment of the invention, the device is configured to be used with multiple user interfaces. For instance, a single WBC reader can pair with multiple user interfaces. In this way, patients bringing in their interface units into e.g. a hospital setting, can readily pair their user interfaces with a single WBC at the care facility. The user interfaces can then be used to identify patients and/or receive information in the care setting either via the WBC reader or through other communication channels.

These and other features of the invention will become apparent from and elucidated with reference to the embodiments described hereinafter.

### BRIEF DESCRIPTION OF THE DRAWINGS

Exemplary embodiments of the invention will be described in the following drawings.

FIG. 1 schematically shows a device for home monitoring of patients according to an exemplary embodiment of the invention.

FIG. 2 schematically shows a device for home monitoring of patients according to another exemplary embodiment of the invention.

FIG. 3 schematically shows a device for home monitoring of patients according to another exemplary embodiment of the invention.

FIG. 4 schematically shows a flow diagram of a method of home monitoring of a patient according to another exemplary embodiment of the invention

In principle, identical parts are provided with the same reference symbols in the figures. Further, the figures are schematic and not in scale.

### DETAILED DESCRIPTION OF EMBODIMENTS

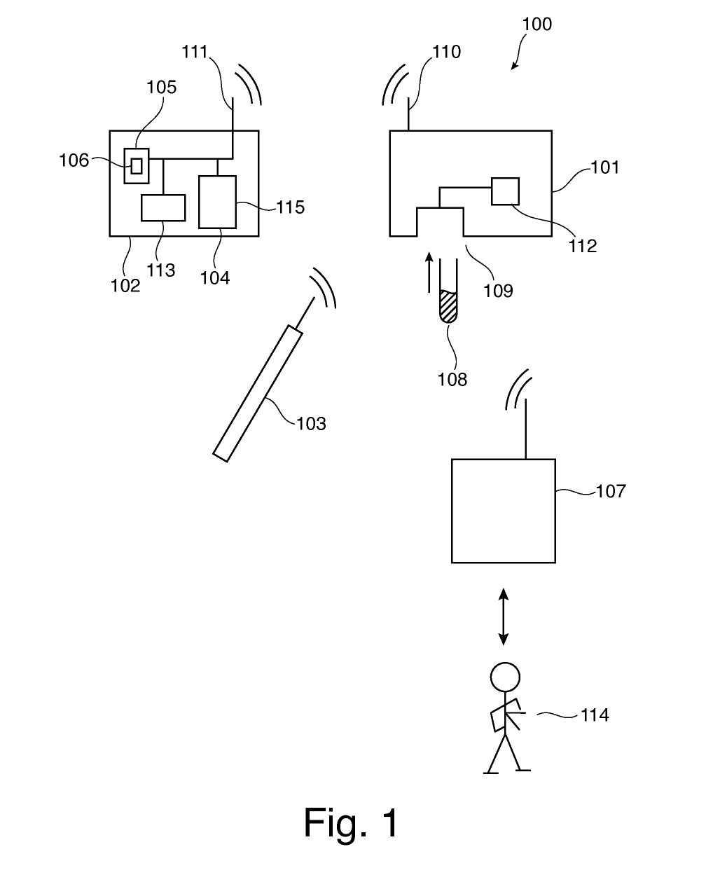

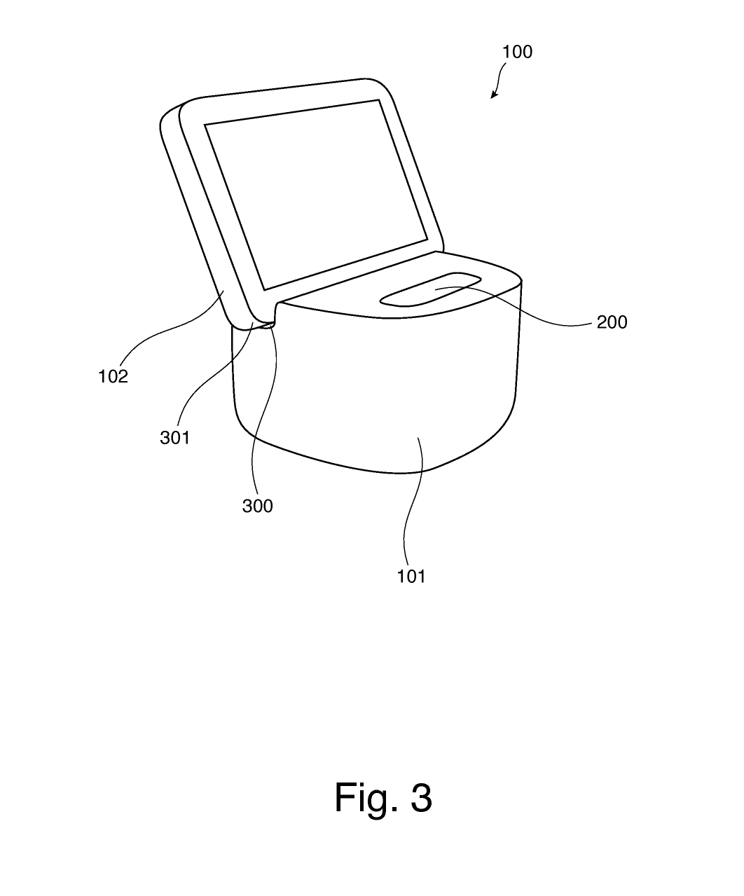

FIG. 1 schematically shows a device 100 for home monitoring of haematological parameters. The device 100 of FIG. 1 comprises a WBC counter 101 and a physically and structurally separated user interface 102. The WBC counter is configured for receiving a sample probe 108, as a receiving slot 109 is provided. The user interface 102 and the cell counter 101 are configured to exchange data with each other. This may be done for example via respective antennas 110 and 111. Different types of transmission techniques may be used for this functionality of both devices. For example, they may be configured to exchange data with each other via at least one element chosen from the group comprising a short-range local radiofrequency transmission, a wide-range radiofrequency transmission, 3G transmission, GPRS transmission, wireless local area network transmission, RFID transmission, infrared transmission, Bluetooth transmission, and any combination thereof. The cell counter 101 may comprise corresponding electronics and sensors 112 to carry out the respective WBC measurement. The user interface 102 comprises a display 104 and an electronical storage medium 105. The display is embodied as a touch screen 115. In the storage medium 105, a health assessment questionnaire 106 is stored to be used by the patient. This questionnaire may be displayed to the patient via display 104. The user interface may also be configured to transmit the completed questionnaire to the clinician 114 via server 107 for example by transmission over antenna 111. In particular, based on inputs from the thermometer 103 or based on inputs from the WBC counter 101, the user interface can ask appropriate questions to the user by questionnaire 106. The embodiment of FIG. 1 comprises a separation of the user interaction device from the cell counter. This allows user interaction away from the cell counter, for example when taking a temperature or completing a questionnaire. It is also possible to add a docking connector to the cell counter as shown in following FIG. 3. This may allow the user interface to be connected to the cell counter and then guide the patient through the steps required to perform the white blood cell counting.

Further, the thermometer 103 in FIG. 1 may be configured to carry out three different temperature measurements per inquiry to get rid of measurement fluctuations. The thermometer may be configured to transmit only the highest of the three measured temperatures to user interface 102. Of course also other numbers, like two, four five, six or more, of different temperature measurements per inquiry are possible.

In a further embodiment all three measurements are sent to the user interface and the user interface will then use the highest of these values for onward transmission to the server. The WBC counter 101 and the thermometer 103 are configured to exchange data with the user interface 102. The communication channel may be unidirectional or bidirectional. There may be various embodiments of the configuration of the thermometer and/or the user interface, such that the determined temperature is provided to the user interface. For example, the user interface may be configured to frequently and electronically read out the thermometer via for example short-range local radiofrequency communication or also via for example a wide-range radiofrequency communication. Also infrared or Bluetooth may be used for transmitting the determined temperature from the thermometer to the user interface. In an analogue way, the thermometer may be configured to frequently transmit, i.e. to send, the determined temperature to the user interface, particularly via Bluetooth. Also a purely automatic procedure may be implemented in the user interface.

The device 100 allows the WBC reader may be in the kitchen or the bathroom, whereas the PRO self assessment via the user interface 102 can take place anywhere in the home, or even on the move. If the user interaction device would be tightly bound to the WBC reader, then the patient is not able to use the device as planned and stored in the user interface 102. Furthermore the clinician might set a schedule for each parameter to be monitored that requires less frequent WBC measurements than other parameters, e.g. through a schedule identified by the WBC trajectory modelling or on-demand when the patient feels unwell. This means that device 100 allows flexibility when a more suitable location for the interaction is needed, e.g. bedside or mobile.

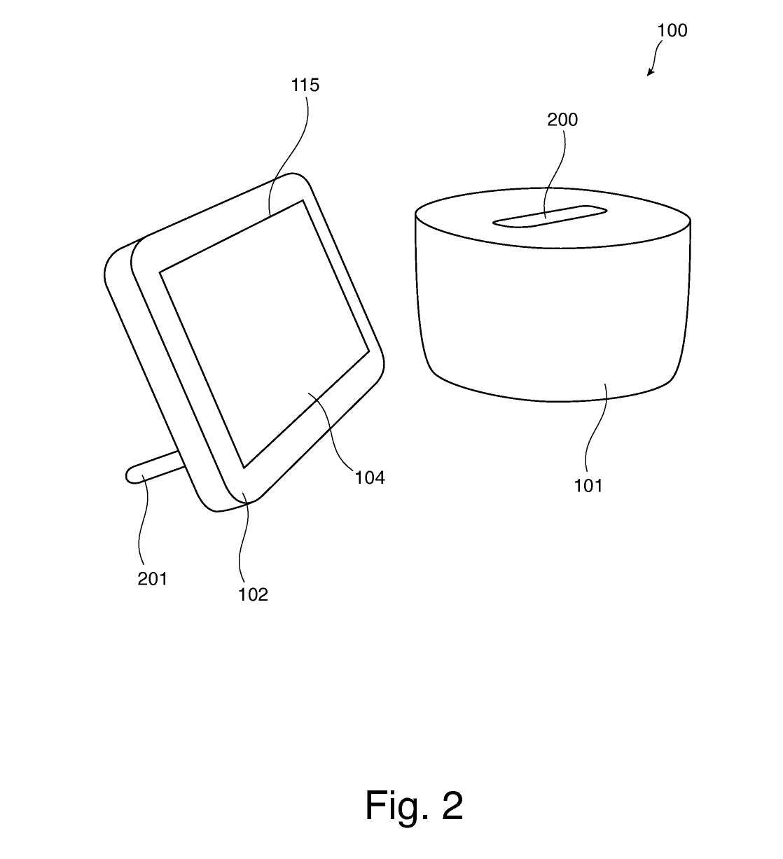

According to another exemplary embodiment of a device for home monitoring of patients, FIG. 2 depicts a WBC counter 101 and a user interface 102. The cell counter 101 comprises a receiving slot 200 in which a sample probe of blood of the patient may be inserted. The user interface 102 comprises a display 104 which is embodied as a touch screen 115. With support member 201 the user interface facilitates a free standing on a desk. The presented separation of the user interface 102 from the WBC counter 101 provides for another advantage. Often, patients undergoing treatment for cancer, require additional treatments on top of their chemotherapy, for example radiotherapy or hormone therapy. In some cases it can be sufficient after chemotherapy to only monitor the temperature and patient reported outcomes, which can be done by the user interface alone. Thus, the presented solution allows the WBC reader to be removed and to be reconditioned after the first treatment (of chemotherapy) and the separate user interface to be left with the patient during subsequent therapies. This may significantly reduce costs during a home monitoring process.

An alternative embodiment is to equip the WBC reader with a docking connector. This allows the remote UI to be charged, to transmit readings to the reader and synchronize the WBC tests with the user interface. The patient can then choose to use the system as a single system or take the interaction device to a more suitable location to complete their questionnaires.

The presented device of FIG. 3 further allows for the following flexibility. Regular or scheduled tests may be pre-determined by the clinician and may be stored within a test calendar on the user interface. The user interface provides for a corresponding configuration. Prior to the scheduled test, an audible and visual prompt can be given to the patient by the user interface reminding him when to perform the test. A non-scheduled test may be performed at any time and can be initiated by the patient pressing an ‘anytime’ button, or the like, on the user interface and then selecting from a list of options the type of test they would like to perform, for example white blood cell count, temperature, or health question. On completion of the test, the results may be automatically sent to the server with clinician and patient messaging operating in a similar fashion to scheduled tests. The patient may elect to perform an anytime test because they feel unwell, or because the clinician has instructed them to perform a non-scheduled test. The device of the present invention thus enhances the flexibility of measurements that are carried out during home monitoring. These advantages may also be achieved by other embodiments of the present invention as a correspondingly adapted user interface may be implemented. Thus, the above aspects do not only apply to the embodiment of FIG. 3.

According to another exemplary embodiment of the invention, FIG. 3 shows a device for home monitoring of patients undergoing chemotherapy in which the WBC counter 101 comprises a first mechanical docking connector 300 for receiving the user interface 102. Moreover, the user interface 102 comprises a second mechanical docking connector 301 for inserting the user interface into the WBC counter. The first mechanical docking connector is embodied as a recess of the cell counter 101 such that the frame or body of the user interface 102 can be engaged with the recess. Consequently, a mechanical fixation of the user interface 102 at the cell counter 101 is reached. However, also other mechanical docking connectors may be used. If desired, an additional electronic connection between the WBC counter and the user interface can be provided in the attached configuration as shown in FIG. 3. Respective electrical connectors and leads are provided by the respective elements.

In the above described embodiments of the present invention the user interface and the WBC counter may be configured to exchange data with each other via at least one element chosen from the group comprising a short-range local radio frequency transmission, a wide-range radio frequency transmission, 3G transmission, GPRS transmission, wireless local area network transmission, RFID transmission, infrared transmission, Bluetooth transmission, and any combination thereof. The same holds true for the thermometer and the user interface.

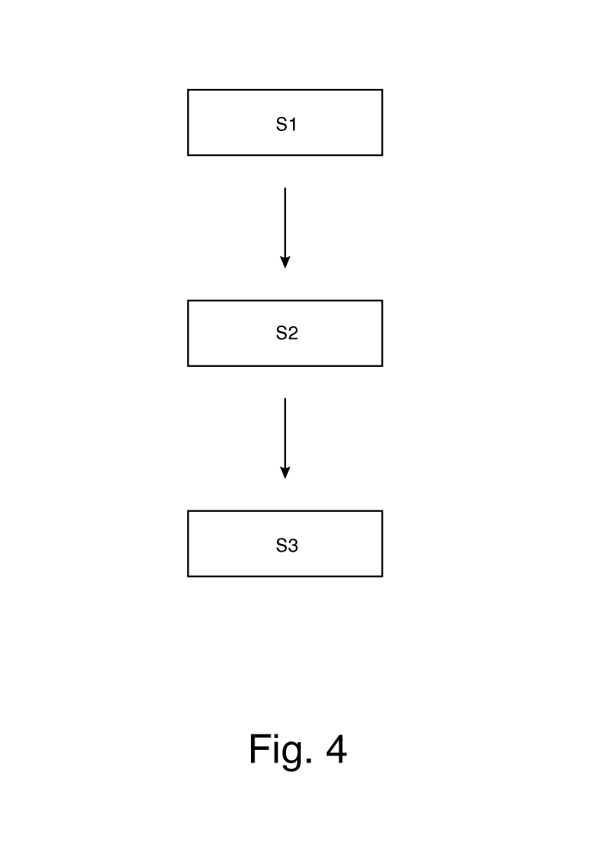

FIG. 4 schematically shows a flow diagram of a method of home monitoring of patients. The method comprising the step determining a value of white blood cells of a sample probe of the patient by a white blood cell counter S1. Further, transmitting the determined value of white blood cells from the white blood cell counter to a physically separated user interface is shown with S2 in FIG. 4. Also the step monitoring the patient by the user interface based on the transmitted value of white blood cells by the user interface S3 is comprised.

Other variations to the disclosed embodiments can be understood and effected by those skilled in the art in practising the claimed invention, from the study of the drawings, the disclosure, and the appended claims. In the claims the word “comprising” does not exclude other elements or steps and the indefinite article “a” or “an” does not exclude a plurality. A single processor or other unit may fulfil the functions of several items or steps recited in the claims. The mere fact that certain measures are recited in mutually different dependent claims does not indicate that a combination of these measures cannot be used to advantage. A computer program may be stored/distributed on a suitable medium such as an optical storage medium or a solid-state medium supplied together with or as part of other hardware, but may also be distributed in other forms, such as via the Internet or other wired or wireless telecommunication systems. Any reference signs in the claims should not be construed as limiting the scope of the claims.

1. Device for home monitoring of haematological parameters of patients, the device comprising:
a white blood cell counter,
a user interface,
wherein the white blood cell counter is configured for receiving a blood sample probe and to determine a value of white blood cells in the received sample probe,
wherein the user interface is physically separated or physically separable from the white blood cell counter, and
wherein the user interface is configured to display information based on the value of white blood cells in the received blood sample probe as determined by the white blood cell counter.

2. Device according to claim 1, further comprising
a thermometer configured to determine a value of a body temperature of the patient, and
wherein the thermometer and the user interface are configured to provide the determined value of the body temperature to the user interface.

3. Device according to claim 2,
wherein the thermometer comprises an interface, in particular a Bluetooth interface, for wireless communication with the user interface.

4. Device according to claim 1,
wherein the white blood cell counter and the user interface are configured to provide the determined value of white blood cells in the received blood sample probe to the user interface.

5. Device according to claim 2,
wherein the user interface stores a white blood cell intervention level,
wherein the user interface stores a temperature intervention level,
wherein the user interface is configured for comparing the provided, determined value of the body temperature with the temperature intervention level, and
wherein the user interface is configured for comparing the determined value of white blood cells in the received blood sample probe with the white blood cell intervention level.

6. Device according to claim 5,
wherein the user interface is configured for generating a signal based on a result of the comparison, and
wherein the signal is chosen from the group comprising a visual alert for the patient, an optical alert for the patient, a sms to a clinician, an email to a clinician, an alert message to a server, and any combination thereof.

7. Device according to claim 1,
wherein the user interface comprises a display and an electronical storage medium, and
wherein a health assessment questionnaire for the patient is stored in the electronical storage medium for displaying the health assessment questionnaire to the patient by the display.

8. Device according to claim 1,
wherein the user interface is configured to transmit information to a remote server.

9. Device according to claim 1,
wherein the white blood cell counter is a display-less device.

10. Device according to claim 1,
wherein the portable white blood cell counter comprises a first mechanical docking connector for establishing a connection with the user interface, and
wherein the user interface comprises a second mechanical docking connector for establishing a connection with the white blood cell counter.

11. Device according to claim 10,
wherein the user interface and the white blood cell counter are configured, in a configuration where the user interface is docked to the white blood cell counter, to charge the user interface by the white blood cell counter, to transmit readings from the user interface to the white blood cell counter, and/or synchronize results from white blood cell counts.

12. Device according to claim 1,
wherein the device is configured to be used with multiple user interfaces.

13. Method of home monitoring of haematological parameters of patients, the method comprising the steps of:
determining a value of white blood cells of a blood sample probe of the patient by a white blood cell counter (S1), and
transmitting the determined value of white blood cells from the white blood cell counter to a physically separated or physically separable user interface (S2),
displaying on the user interface information based on the value of white blood cells in the received blood sample probe as determined by the white blood cell counter (S1).

14. Method according to claim 13, the method comprising the step of:
receiving a value of a body temperature of the patient by the user interface from a thermometer (S4).

15. Method according to claim 14,
wherein the user interface stores a white blood cell intervention level,
wherein the user interface stores a temperature intervention level,
comparing the provided, determined value of the body temperature with the temperature intervention level by the user interface, and
comparing the determined value of white blood cells in the received blood sample probe with the white blood cell intervention level by the user interface.

16. Method according to claim 15,
generating a signal based on a result of the comparison by the user interface, and wherein the signal is chosen from the group comprising a visual alert for the patient, an optical alert for the patient, an sms to a clinician, an email to a clinician, an alert message to a server, and any combination thereof.
